(19) United States
(12) Patent Application Publication
(10) Pub. No.: US 2018/0344156 A1
(43) Pub. Date: Dec. 6, 2018

(54) Ophthalmic Observation/Illumination Set for Adjusting Illumination Conditions and Working Distance

(71) Applicant: Arkadiy Farberov, Newark, CA (US)

(72) Inventor: Arkadiy Farberov, Newark, CA (US)

(21) Appl. No.: 15/613,056

(22) Filed: Jun. 2, 2017

(51) Int. Cl. A61B 3/14; A61B 3/107; G02B 21/06

(52) U.S. Cl. CPC A61B 3/14; G02B 21/0012; G02B 21/06; A61B 3/107

(57) ABSTRACT

Provided is an ophthalmic observation/illumination set for adjusting illumination conditions and a working distance to the object of observation and illumination. set consists of a main optical lens and a couple of interchangeable illumination members that can be assembled in various combinations with the main optical lens so that a light emitted from one of the illumination members will intersect the optical element of the main optical lens or will pass to the object around the optical element of the main optical lens. The set also contains a common handle with batteries for supply of power to the illumination member connected to the main optical lens and an additional optical component such as a Barlow lens selected from a set of Barlow lenses of different curvatures or a fisheye lens.

### FIELD OF THE INVENTION

The invention relates to the field of ophthalmic instruments, in particular to an ophthalmic observation/illumination set for adjusting illumination conditions and a working distance. More specifically, the invention relates to the aforementioned ophthalmic set for observation of a human eye for diagnostics or during surgery with possibility of optimization of illumination conditions and selection of a working distance most optimal for the observation.

### BACKGROUND OF THE INVENTION

Ophthalmic lenses for observation of the human eye interior is an optical instrument that finds wide application in the field of ophthalmology for inspection of conditions in the eye interior, e.g., on the retina, in the vitreous body, etc. One important requirement associated with the practical use of ophthalmic lenses for indirect observation of the eye interior is efficient illumination of the area to be observed.

There exists a variety of optical instruments for illumination of the eye interior during observation through a hand-held ophthalmic lens placed in front of the patient's eye. In a majority of cases, ophthalmologists use for observation of the eye interior illumination emitted from so-called slit lamps or from illuminators secured to the head of the viewer, or so on.

In a conventional ophthalmic procedure, an ophthalmologist observes the interior of the eye by using a slit lamp, which is an instrument consisting of a high-intensity light source that can be focused to shine a thin strip of light into the eye. It is used in conjunction with an ophthalmic lens. The lamp facilitates an examination of the anterior segment and posterior segment of the human eye, which includes the eyelid, sclera, conjunctiva, iris, natural crystalline lens, and cornea. The ophthalmic lenses, which are often called also as bio-lenses, are manufactured in a great variety of types, dimensions, and characteristics and their front focal distances may correspond to dioptric values (D values) in the range of D=5 to D=40, or even greater. Lenses of high dioptric values (D=40; 60; 90) are used with illumination only by slit lamps.

The illumination is carried out through a completely open pupil of the eye and for this purpose the pupil is preliminarily dilated. It is understood that the result of the observation will greatly depend on the illumination conditions of the eye interior. In such a procedure, the slit lamp is normally attached to the viewer's head.

With recent development of the illumination technique and, in particular, with appearance of such light sources as light emitting diodes (LEDs), new avenues were open for application of the LEDs in ophthalmology. In particular, some lenses provided with self-illumination means arranged around the periphery of the lens found practical use.

For example, U.S. Pat. No. 6,547,394 issued on Apr. 15, 2003 to V. Doherty discloses an ophthalmic illuminator including a battery, an electrical resistor, an electrical switch and a plurality of circumferentially arranged LEDs. An electrical switch is in circuit with the resistor, where electrical energy flows through the circuit when the switch is in the closed position. The LEDs are in circuit with the switch. An optical element (e.g., a lens or reflector) can be disposed between the diodes and the eye being observed for illuminating the eye during the observation. The user views the patient's eye through the center of the housing.

U.S. Pat. No. 8,740,383 issued on Jun. 3, 2014 to P. Yates discloses a self-illuminated handheld lens for retinal examination and photography and a related method. The method is aimed at providing a wider field of view of the retina and at eliminating those lens reflections that result from external slit illumination of the handheld condensing lens. The ideal illumination for the retina is a ring of light focused on the eye with a diameter slightly less than the pupil diameter.

Next, U.S. Pat. No. 7,048,379 issued May 23, 2006 to J. Miller, et al., discloses an imaging lens and an illumination system for a retinal camera. Miller's ring illumination is focused on the patient's retina through a front objective lens. The lighting is located behind the objective lens, and the camera is not designed with a contact lens.

### SUMMARY OF THE INVENTION

The invention relates to the field of ophthalmic instruments, in particular to an ophthalmic observation/illumination set for adjusting illumination conditions and a working distance. More specifically, the invention relates to the aforementioned ophthalmic set for observation of a human eye with possibility of optimization of illumination conditions and selection of a working distance most optimal for observation.

The ophthalmic lens set of the invention consists of a number of interchangeable components that can be assembled in various combinations for forming sub-assemblies most optimal for observation conditions with backlight illumination, frontlight illumination, and/or an optimized working distance. The set includes plurality of circumferentially arranged light sources such as light emitting diodes (hereinafter referred to as LEDs) and is provided with a common handle that can be connected to various components and contains batteries for supplying power to the LEDs.

### BRIEF DESCRIPTION OF THE DRAWINGS

FIG. 1A is a front view of an ophthalmic main optical lens of the invention.

FIG. 1B is a rear view of a backlight collar of the invention that supports LEDs and is shown connected to a common handle.

FIG. 1C is a rear view of a frontlight collar of the invention.

FIG. 1D is a front view of one of replaceable Barlow lenses of the invention the replacement of which may change a distance from the patient's eye to the ophthalmic lens in an assembled state of the ophthalmic illumination/observation instrument.

FIG. 1E is a front view of a fisheye lens used in the ophthalmic set of the invention.

FIG. 2 is a perspective view of the common handle which contains batteries for supplying energy to light sources of the set components.

FIG. 3A is a three-dimensional view of the back-light collar of FIG. 1B.

FIG. 3B is a three-dimensional of a sub-assembly of the handle with the backlight collar and the main lens.

FIG. 3C is a longitudinal sectional view of the sub-assembly of FIG. 3B.

FIG. 4A is a longitudinal sectional view of a sub-assembly of an ophthalmic observation/illumination device, which consists of the backlight collar that holds the main ophthalmic lens and a Barlow lens.

FIG. 4B is a front view of a Barlow lens of the invention.

FIG. 4C is a side view of the Barlow lens of FIG. 4B.

FIG. 4D is a view similar to FIG. 4A with the Barlow lens shifted axially in the direction of axis O-O to the right from its position in FIG. 4A (i.e., away from the viewer).

FIG. 5 is a longitudinal sectional view of a sub-assembly that consists of the main optical lens and the frontlight collar.

FIG. 6 is a longitudinal sectional view of a sub-assembly that consists of the main optical lens, the frontlight collar, and the Barlow lens.

FIG. 7 is an axial sectional view of a modified frontlight collar with flexible light-emitting diode holders.

FIG. 8 is a view similar to FIG. 6 wherein a fisheye optical lens is used instead of a Barlow lens.

### DETAILED DESCRIPTION OF THE INVENTION

The invention relates to the field of ophthalmic instruments, in particular to an ophthalmic observation/illumination set for adjusting illumination conditions and a working distance. More specifically, the invention relates to the aforementioned ophthalmic set for observation of a human eye with possibility of optimization of illumination conditions and selection of a working distance most optimal for observation. Furthermore, the ophthalmic set of the invention makes it possible to significantly improve the quality of the eye observation by increasing brightness and contrast on the object being observed.

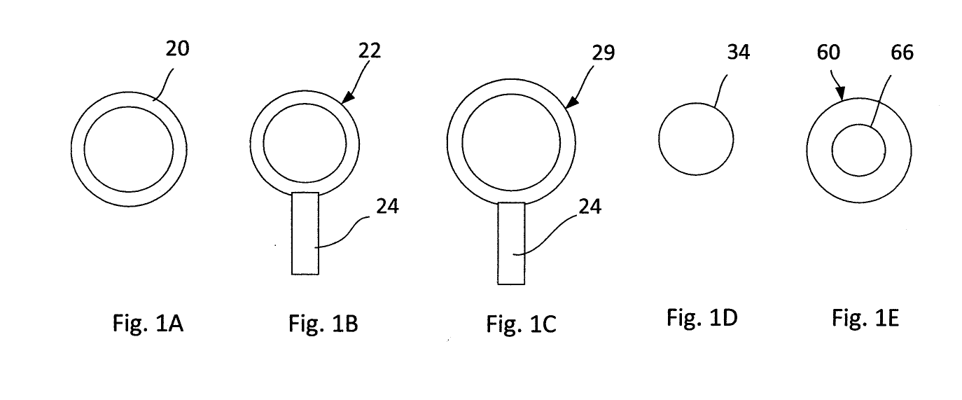

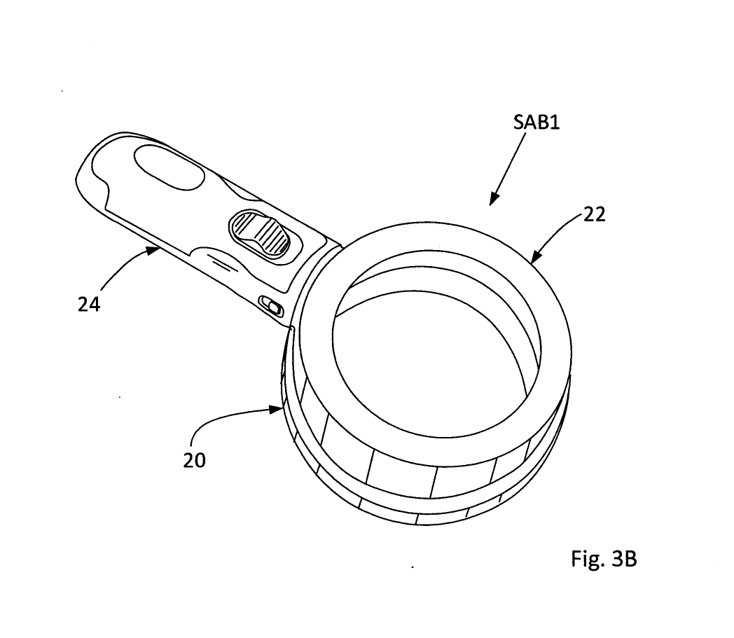

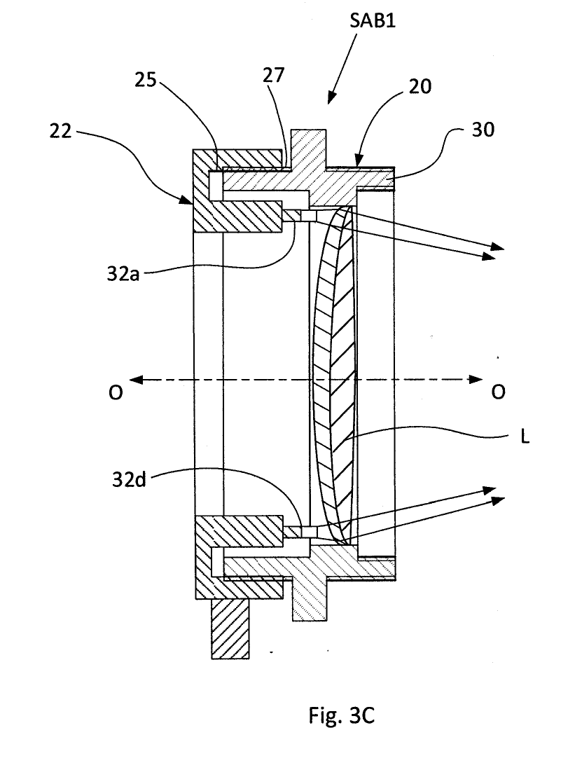

The invention will now be described in more details with reference to the accompanying drawings, wherein FIGS. 1A, 1B, 1C, 1D, and 1E are components of the ophthalmic observation/illumination set that can be assembled in various combinations for improving versatility of the ophthalmic instrument and providing optimization of the eye observation conditions. In the following drawings, the front view is a view from the side of a patient, and rear view is a view from the side of a viewer. More specifically, FIG. 1A is a front view of an ophthalmic main optical lens per se, FIG. 1B is a rear view of a back-light collar 22 that supports LEDs and is shown connected a common handle 24 that contains batteries (not shown) for feeding the LEDs (not seen in FIG. 1B). FIG. 3 is rear view of the backlight collar that supports the LEDs and is shown connected a common handle 24 that contains batteries (not shown) for feeding the LEDs. FIG. 1C is a rear view of a frontlight collar 29 with light sources (not shown in FIG. 1C). FIG. 1D is a front view of one of replaceable Barlow lenses 34, replacement of which may change the working distance. In the context of the present specification the term “working distance” is a distance from the patient's eye to the ophthalmic lens 20 in an assembled state of the ophthalmic illumination/observation instrument. FIG. 1E is a front view of a fisheye lens 60.

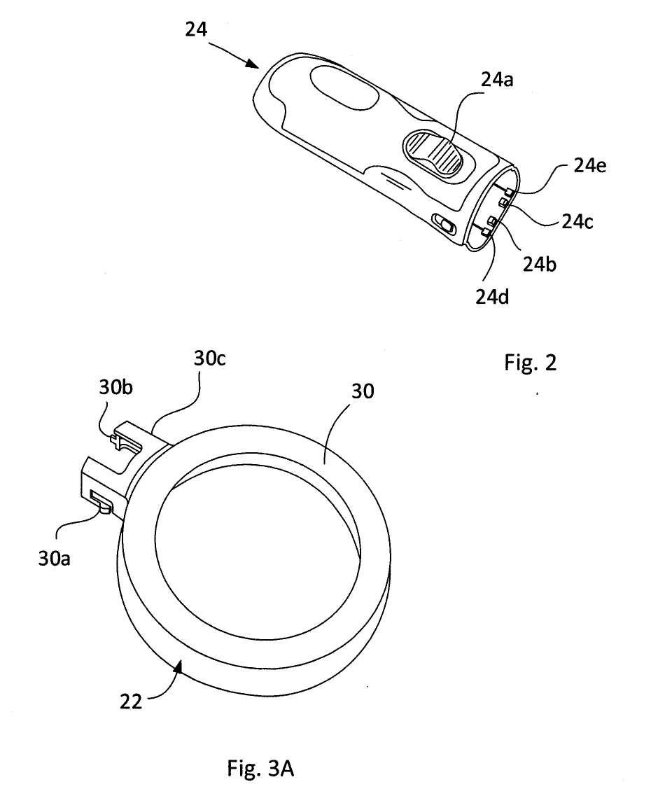

FIG. 2 is a perspective view of the common handle 24 which contains batteries (not shown) conventionally used in similar handles of lenses as a source of illumination of an object being observed (see, e.g., Reizen Maxi-Brite illuminating Stand Magnifier 5× −2.4 inch −60 MM Lens). The handle 24 has a battery ON/OFF switch 24a and output contacts 24b and 24c compatible with receptacles of the backlight collar 22 and the frontlight collar (not shown in FIG. 2). Reference numerals 24d and 24e designate latches engageable with respective spring-loaded stoppers 30a and 30b, which are formed in a radial projection 30c from a main-lens holder rim 30 shown in FIG. 3A. This drawing is a three-dimensional view of a backlight collar 22. A similar handle-connection mechanism (not shown) for connection of the common handle 24 is provided on the frontlight collar 29.

Having briefly described components of the ophthalmic observation/illumination set, let us consider various combinations of the set for providing most optimal illumination and/or observation conditions.

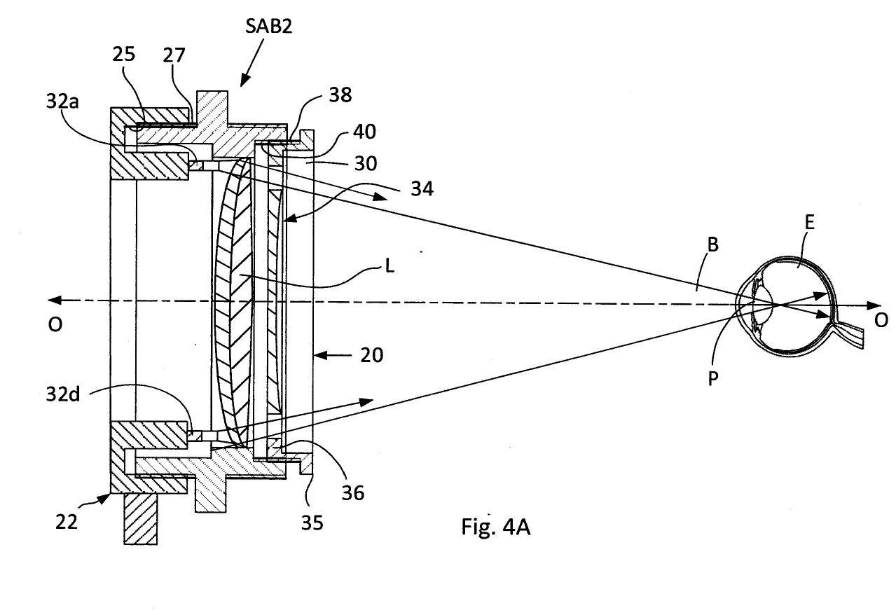

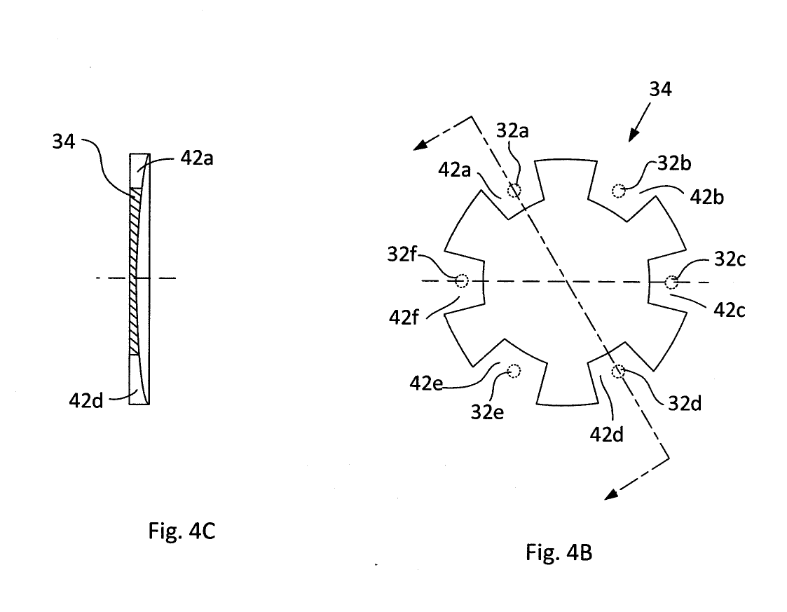

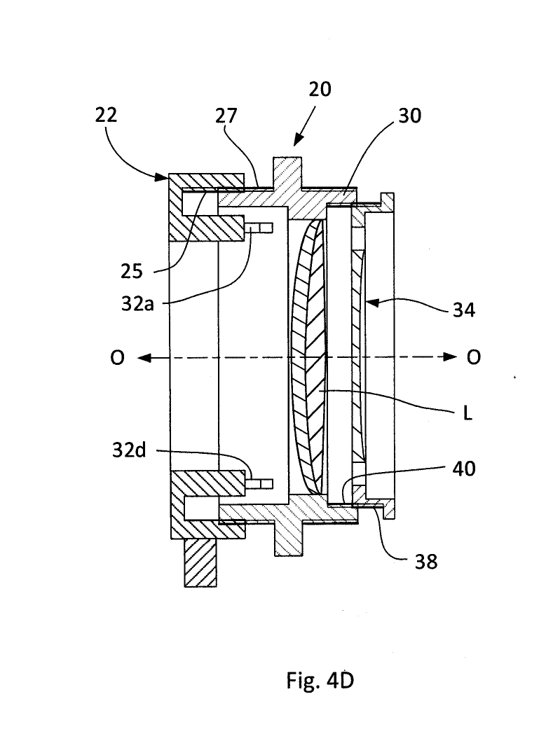

FIG. 3B is a three-dimensional view of a sub-assembly SAB1 consisting of the handle 24 with the backlight collar 22 and the main lens 20. The longitudinal sectional view of the sub-assembly SAB1 is show in FIG. 3C. It can be seen that the backlight collar 22 holds the main ophthalmic lens 20. The backlight collar 22 has an inner thread 25 which engages an outer thread 27 formed on a main-lens holder rim 30 which holds an ophthalmic lens element L. The threaded connection between the backlight collar 22 and the main-lens holder rim 30 provides axial movement of the backlight collar 22 relative to the main lens 20 in the direction of the central optical axis O-O. The rim 30 supports a plurality of circumferentially arranged illumination elements, e.g., light emitting diodes (LEDs) 32a, 32b, 32c, 32d, 32e, and 32f, only two of which, i.e., LEDs 32a and 32d are shown in FIG. 3C. The rest are seen in FIG. 4B which is explained later. The LEDs are arranged circumferentially and concentrically to the main optical lens element L.

FIG. 4A is a longitudinal sectional view of a sub-assembly SAB2 of an ophthalmic observation/illumination device, which consists of the backlight collar 22 that holds the main ophthalmic lens 20 and a Barlow lens 34. Those parts of the sub-assembly SAB2 which constitute the sub-assembly SAB1 shown in FIG. 3C will be omitted from the description of the sub-assembly SAB2 and only their designation will remain in FIG. 4A.

More specifically, although the structure of the main ophthalmic lens 20 with the lens element L and with the backlight collar 22 that holds light emitting diodes (LEDs) such as 32a, 32d, etc. which emit light beams that pass through the periphery of the lens element L, can be used as is, the inventor herein made it possible to incorporate into the sub-assembly SAB1 an additional optical element, e.g., an additional optical lens such as the so-called Barlow lens 34 which allows to adjust in some range the dioptric value D of the device and to select those dioptric values which are most appropriate for each specific condition. The Barlow lens 34 is held by a Barlow lens holder 36, which is threaded with its outer thread 38 into an inner thread 40 formed on the front inner surface of the lens holder rim 30.

In FIG. 4A the sub-assembly SAB2 is shown in an optical circuit that illustrates passage of the light beam B from the backlight LEDs 32a, 32b, 32c, 32d, 32e, and 32f, the lens L, and the pupil P of the eye E to the eye retina. It is understood that within the eye the optical rays are shown very schematically and that in reality the optical circuit formed within the eye is much more complicated in its nature.

The Barlow lens 34 is a diverging lens, which is used in series with other optics in optical systems and increases the effective focal length of all preceding optical components. A practical result is that inserting of a Barlow lens magnifies the image.

A basic requirement to the main ophthalmic lens 20 is to provide a very high quality in transmission of the image from the object to the eye of the viewer (not shown). This is achieved by either assembling the main ophthalmic lens 20 from two specially selected lens components with different refraction indices and/or by providing the lens 20 or lens components with properly chosen aspheric surfaces.

For not blocking the light passing through the main ophthalmic lens 20 to the eye E (see FIG. 4A) of the patient, the Barlow lens 34 is a weak-negative lens that has a special configuration in a front view shown in FIG. 4B with recesses 42a, 42b, 42c, 42d, 42e, and 42f the positions of which during the procedure are aligned with the position of the LEDs 32a, 32b, 32c, 32d, 32e, and 32f. In a simplified form the indexing means may be formed as marking lines on the flange 35 of the Barlow lens holder 36 and an alignment mark (not shown) on the rim 30 of the mail lens, which is maintained stationary during the procedure. For convenience of handling, the parts which are grasped by the user's fingers may be provided with serrations (not shown).

FIG. 4C is a side view of the Barlow lens 34.

FIG. 4D is a view similar to FIG. 4A with the Barlow lens 34 shifted axially in the direction of axis O-O to the right from its position on FIG. 4A (i.e., away from the viewer). Furthermore, in FIG. 4D, the LEDs 32a, 32d, etc. are shown shifted axially to the left in the direction of axis O-O from the position shown in FIG. 4A for adjusting the illumination conditions in observation of the patient's eye E, i.e., for obtaining the most optimal brightness of the illumination spot in the eye interior, e.g., on the eye retina. In FIG. 4D the sub-assembly SAB2 is shown separately, i.e., without illustration of the passage of the light beams in the optical system.

The Barlow lens 34 is also used for obtaining the most optimal focal length of the entire optical sub-assembly SAB2 that may contain the Barlow lens with adjustment of the lens position in the direction of the optical axis.

The Barlow lenses 34 may be replaceable and removed or connected by unscrewing the threaded portion 38 of the Barlow lens holder 36 from the inner thread 40 of the main-lens holder rim 30. Alternatively, the Barlow lenses may be insertable into a slot or pivotally attached to the holder 36. The pivot and slots are not shown.

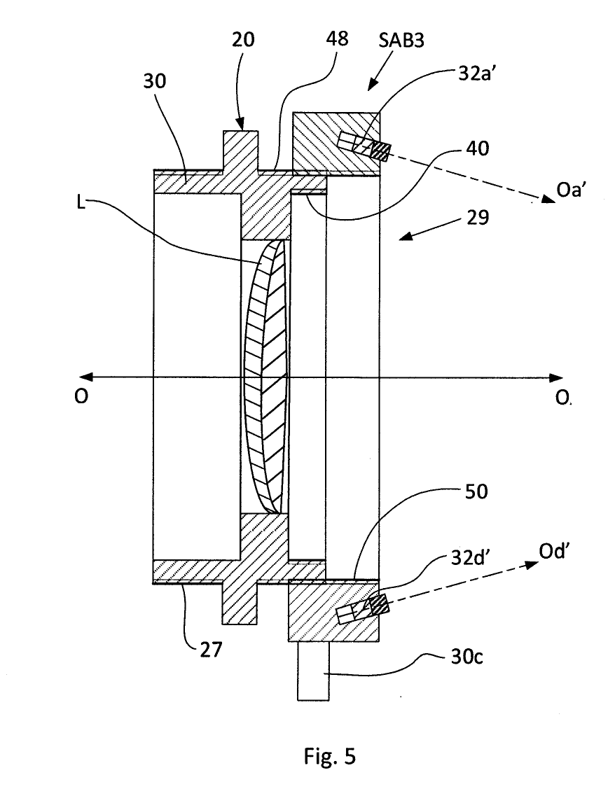

FIG. 5 is a longitudinal sectional view of a sub-assembly SAB3 that consists of the main optical lens 20 and the frontlight collar 29. The main optical lens 20 is the same device of the set that was shown in connection with sub-assemblies SAB1 and SUB2 and therefore a description thereof is omitted. The frontlight collar 29 has an inner thread 50 engageable with an outer thread 48 which is formed on the side of the main-lens holder rim 30 opposite to the thread 27 for engagement with the backlight collar 22 that may be connected to the lens holder 30 from the opposite side.

On its front side, which in use faces the patient, the frontlight collar 29 supports a plurality of light sources such as LEDs (which may be the LEDs of the same type as those shown in FIGS. 4A, 4B, and 4D and only two of which, i.e., the LEDs 32a′ and 32d′, are shown in FIG. 5). These LEDs have their optical axes such as O_{a′} and O_{d′} tilted inward for forming tapering beams which have their focus point in the pupil of the patient's eye (not shown in FIG. 5). In this subassembly, the light emitted by the light sources 32a′, 32d′, etc. passes to the patient's eye above the lens edges without optically intersecting the lens element L. The frontlight collar 29 has an inner thread 50, which is engageable with the outer thread 48 formed on the front side of the main-lens holder rim 30. Similar to the backlight sub-assemblies, intensity of light that illuminates the object in the sub-assembly SUN3 can be adjusted by twisting or untwisting the frontlight collar 29 on the thread 48 of the main-lens holder rim 30 thus optimizing the illumination conditions on the object being observed.

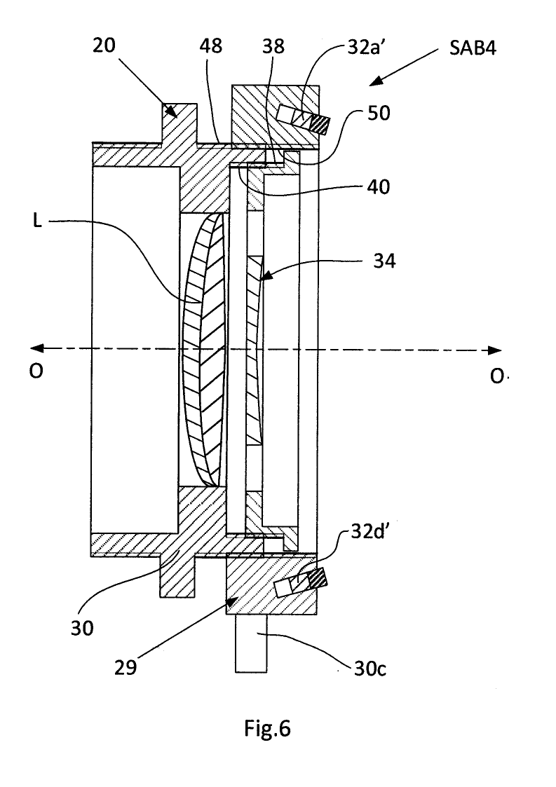

FIG. 6 is a longitudinal sectional view of a sub-assembly SAB4 that consists of the main optical lens 20, the frontlight collar 29, and the Barlow lens 34. The main optical lens 20 is the same device of the set that was shown in connection with sub-assemblies SAB1, SUB2, and SUB3 and therefore the description thereof is omitted. By moving the frontlight collar 29 on the thread 48 of the rim 30, it is possible to change the position of the focusing point (not shown) of converging light beams (FIG. 5) emitted from the LEDs 32a′, 32d′, etc. This is important not only from viewpoint of adjusting the light intensity of the light spot but also in view of replaceability of the Barlow lenses, which, as mentioned earlier, are available in a set. Each Barlow lens of the set will have a different surface curvature and may require adjustment in the direction of the optical axes for matching to the most optimal observation and illumination conditions. Similar to the case of the backlight collar, the LEDs 32a′, 32d′ of the frontlight collar 29 are connectable via the radial projection 30c to the power supply batteries contained in the handle 24.

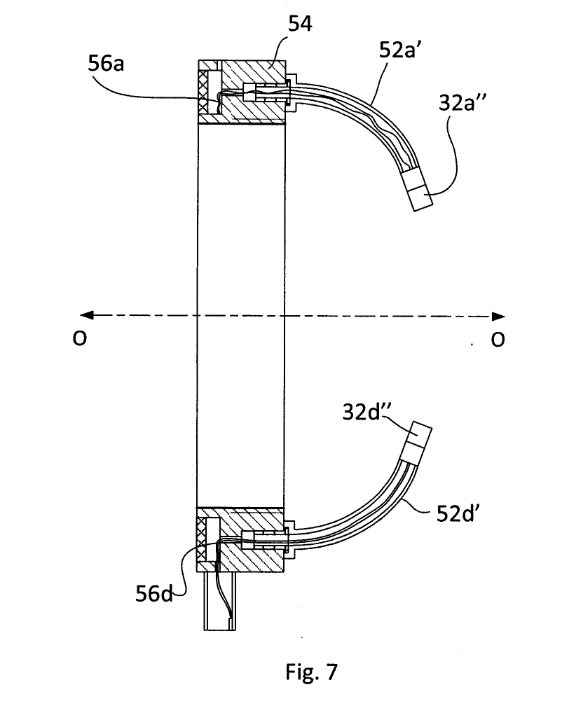

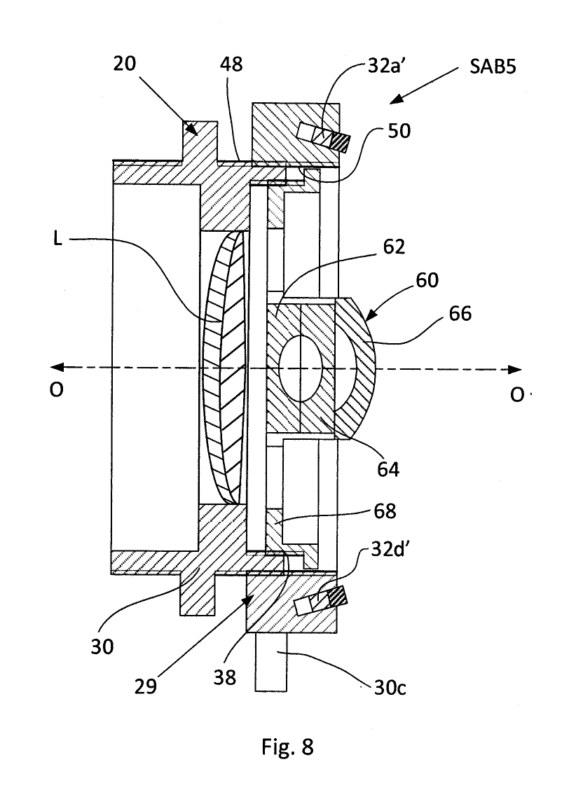

According to one or several aspects of the invention and as shown in FIG. 7, the frontlight LEDs 32a″, 32d″, etc. may be held by front ends of the flexible gooseneck arms 52a′, 52d′, etc., the rear ends of which are fixed to the end faces of the frontlight collar 54. This collar 54 has the same inner thread as the thread 50 (FIG. 8) of the frontlight collar 29 for engagement with the outer thread 48 of the main optical lens 20.

The flexible gooseneck light source arms 52a′, 52d′, etc., are arranged circumferentially around and above the main lens element L and support respective light sources, i.e., LEDs 32a″, 32d″, etc., at the ends of the arms 52a′, 52d′, etc.

In FIG. 7, reference numerals 56a, 56d, etc. show contact terminals for respective contacts of the feeding power supply batteries (e.g., batteries AAA not shown) placed into the common handle 24 (FIG. 2). It is understood that due to the provision of the gooseneck arms, the LEDs may be arranged at different angle and at different distances from the object of the illumination, i.e., the patient's eye.

The flexible gooseneck arms for supporting light sources are especially important for illumination of the patient's eye when the set is combined into a sub-assembly SAB5 composed of the main lens 20, backlight collar 22 or frontlight collar 29 and an additional optical element such as a fisheye lens 60 (FIG. 8) which is used instead of the Barlow lens 34. In other words, the fisheye lens constitutes an additional optical element that can be used instead of the Barlow lens in the same combinations as the Barlow lens with other set components. FIG. 8 is a sectional view of the SAB5. Those components of the assembly SAB5 which remain the same as in the previously described sub-assemblies are designated by the reference numerals as above. More specifically, the main-lens holder rim 30 supports the main optical lens element L and the frontlight collar 29 (or frontlight collar 54, if the modified illuminator of the type shown in FIG. 7 is used). In FIG. 8, reference numeral 60 designates a fisheye lens. A fisheye lens is a specific optical lens with abnormally large aperture which may be as big as 180°. The fisheye 60 used in the set of the present invention may have a composite structure that as an example may consist of two oppositely symmetrical negative lenses 62 and 64 in combination with a front positive lens that has a small radius of curvature and hence large curvature. The fisheye lens 60 is held by a fisheye holder 68 which has the same thread 38 as the Barlow lens holder 36 for engagement with the inner thread 40 of the main-lens holder rim 30.

Thus, it has been shown that the ophthalmic observation/illumination set of the present invention constitutes a universal ophthalmic instrument that may be formed into different sub-assemblies for obtaining the most optimal observation and illumination conditions needed for performing an ophthalmic procedure. This becomes possible by combining the main optical lens 20 in various combinations with the backlight collar 22, frontlight collar 29, and the Barlow lens 34.

Furthermore, it was shown that the ophthalmic observation/illumination set of the invention makes it possible to adjust illumination conditions and working distances to an object of observation and illumination. The main optical lens 20 contains the optical lens element L and has a first connection means (threads 46 and 48) for connecting a first component of the set (the frontlight collar 29), a second connection means (threads 25 and 27) for connecting a second component of the set (the backlight collar 22), and a third connection means (the spring-loaded stoppers 30a and 30b) for connecting a third component of the set (the handle 24), wherein the first component of the set has a frontlight illumination source (the LEDs), the second component of the set is a backlight illumination source (other LEDs), and the third component of the set is an interchangeable power source (the handle with the batteries) for supplying energy to the frontlight illumination source or the backlight illumination source, depending on which one is connected to the main optical rim 30, wherein the first component of the set, the second component of the set, and the third component of the set may form sub-assemblies, and wherein in a sub-assembly composed of the backlight illumination source and the main optical lens a light emitted from the backlight illumination source passes to the object through the optical lens body, and wherein in a sub-assembly composed of the main optical lens and the frontlight illumination source a light emitted from the frontlight illumination source passes to the object around the optical lens element.

Although the invention has been shown and described in detail with reference to specific examples and combinations of the components into various sets, it is understood that these examples should not be construed as limiting the applications of the invention and that various changes and modifications are possible without deviation from the scope of the attached patent claims. For example, the number of the LEDs may be different from the six shown in FIG. 4B. The LEDs may emit lights of different wavelengths for providing beams of different colors. The backlight collar can be provided with screens for limiting the access of the scattered and reflected light to the patient's eye. The threaded connections of the set components were shown as threaded connections only as examples, and fixable sliding connections on splines may be used instead. The fisheye structure shown in FIG. 8 was shown only as an example and other fisheye lens modifications are possible.

What I claim is:

1. An ophthalmic observation/illumination set for adjusting illumination conditions and working distance to an object of observation and illumination comprising:
a main optical lens that comprises an optical lens element and has a first connection means for connecting a first component of the set, a second connection means for connecting a second component of the set, and a third connection means for connecting a third component of the set, wherein the first component of the set is a frontlight illumination source, the second component of the set is a backlight illumination source, and the third component of the set is an interchangeable power source for supplying energy to the frontlight illumination source or the backlight illumination source depending on which one is connected to the main optical lens holder, wherein the first component of the set, the second component of the set, and the third component of the set may form sub-assemblies, and wherein in a sub-assembly composed of the backlight illumination source and the main optical lens a light emitted from the backlight illumination source passes to the object through the optical lens element, and wherein in a sub-assembly composed of the main optical lens and the frontlight illumination source a light emitted from the frontlight illumination source passes to the object around the optical lens element.

2. The ophthalmic observation/illumination set according to claim 1, wherein the first illumination means and the second illumination means comprise pluralities of light emitting diodes arranged circumferentially and concentrically to the main optical lens.

3. The ophthalmic observation/illumination set according to claim 2, wherein the main optical lens has an optical axis and wherein the first connection means and the second connection means comprise threaded connections that allow axial displacement of backlight illumination source and the frontlight illumination source in the direction of the optical axis.

4. The ophthalmic observation/illumination set according to claim 3, wherein the main optical lens further comprises a fourth component of the set and a fourth connection means for connecting the fourth component.

5. The ophthalmic observation/illumination set according to claim 4, wherein the fourth component is an additional optical lens.

6. The ophthalmic observation/illumination set according to claim 5, wherein the additional optical lens is at least one Barlow lens.

7. The ophthalmic observation/illumination set according to claim 3, wherein the main optical lens further comprises a fourth connection means for connecting a fourth component, which is a Barlow lens selected from a set of Barlow lenses of different curvature.

8. The ophthalmic observation/illumination set according to claim 6, wherein the Barlow lens has a plurality of recesses aligned with the positions of the light emitting diodes of the first illumination sources for passing the light emitted by the light emitting diodes without optically intersecting the Barlow lens.

9. The ophthalmic observation/illumination set according to claim 3, wherein the main optical lens comprises a lens holder rim that holds the main lens element and has a first outer thread on one side and a second outer thread a on the other side.

10. The ophthalmic observation/illumination set according to claim 9, wherein the backlight illumination means comprises an inner thread for threaded engagement with the first outer thread of the lens holder rim.

11. The ophthalmic observation/illumination set according to claim 9, wherein the frontlight illumination means comprises an inner thread for threaded engagement with the second outer thread of the lens holder rim.

12. The ophthalmic observation/illumination set according to claim 11, wherein the light emitting diodes of the second illumination means have optical axes in the direction of the light emitted by the light emitting diodes of the second illumination means, wherein the optical axis of the light emitting diodes are titled inward in a tapered manner relative to the optical axis of the main optical lens.

13. The ophthalmic observation/illumination set according to claim 9, wherein the lens holder rim further comprises an inner thread on the side of the second outer thread, and wherein the Barlow lens comprises an outer thread for threaded engagement with the inner thread of the lens holder rim on the side of the second outer thread.

14. The ophthalmic observation/illumination set according to claim 13, wherein the Barlow lens has a plurality of recesses aligned with the positions of the light emitting diodes of the first illumination sources for passing the light emitted by the light emitting diodes without optically intersecting the Barlow lens.

15. The ophthalmic observation/illumination set according to claim 5, wherein the additional optical lens is a fisheye lens.

16. The ophthalmic observation/illumination set according to claim 1, wherein the frontlight illumination source comprises a plurality of flexible gooseneck light source arms arranged circumferentially around and above the main lens and respective light sources supported by each gooseneck light source holder.

17. The ophthalmic observation/illumination set according to claim 16, wherein light sources are light emitting diodes.

18. The ophthalmic observation/illumination set according to claim 5, wherein the frontlight illumination source comprises a plurality of flexible gooseneck light source arms arranged circumferentially around and above the main lens and respective light sources supported by each gooseneck light source holder.

19. The ophthalmic observation/illumination set according to claim 18, wherein light sources are light emitting diodes.
